(19) United States
(12) Patent Application Publication
(10) Pub. No.: US 2018/0055339 A1
(43) Pub. Date: Mar. 1, 2018

(54) INFLATABLE COVERS FOR TRANSESOPHAGEAL ECHOCARDIOGRAPHY TRANSDUCER PROBES

(71) Applicant: David R. RICHARDS, New Albany, OH (US)

(72) Inventor: David R. RICHARDS, New Albany, OH (US)

(21) Appl. No.: 15/689,014

(22) Filed: Aug. 29, 2017

Related U.S. Application Data
(60) Provisional application No. 62/380,819, filed on Aug. 29, 2016.

(51) Int. Cl. A61B 1/00; A61B 8/12; A61B 8/00; A61M 25/10; A61B 8/08; A61M 39/22

(52) U.S. Cl. CPC A61B 1/00082; A61B 8/12; A61B 8/445; A61B 1/00135; A61M 25/10185; A61M 2039/229; A61B 8/0883; A61M 39/22; A61M 2025/1072; A61M 2025/1061; A61B 1/0014

(57) ABSTRACT

A flexible cover for a probe having an ultrasound emitting tip that includes an inflatable portion, wherein the inflatable portion further includes a first reservoir, wherein the first reservoir is adapted to fit over the ultrasound emitting tip of the probe, and wherein the first reservoir is adapted to receive a first predetermined volume of fluid; a second reservoir adjacent to the first reservoir, wherein the second reservoir is adapted to receive a second predetermined volume of fluid; and a divider for completely separating the first reservoir from the second reservoir; and a collar connected to or formed integrally with the inflatable portion, wherein the collar is operative to seal the inflatable portion against the probe.

### CROSS-REFERENCE TO RELATED APPLICATIONS

This patent application claims the benefit of U.S. Provisional Patent Application Ser. No. 62/380,819 filed on Aug. 29, 2016 and entitled “Inflatable Covers for Transesophageal Echocardiography Transducer Probes”, the disclosure of which is hereby incorporated by reference herein in its entirety and made part of the present U.S. utility patent application for all purposes.

### BACKGROUND OF THE INVENTION

The described invention relates in general to ultrasound imaging systems, devices, and accessories, and more specifically to a contact-enhancing condom or cover that fits snugly over one end of a transesophageal echocardiography (TEE) transducer probe.

Transesophageal echocardiography (TEE) is an ultrasound imaging modality that provides real-time images of the heart for diagnostic and therapeutic procedures. TEE provides superior image quality compared to standard transthoracic echo tests that are conducted from outside a patient's body. TEE can provide a detailed view of a patient's heart valves and chambers because the TEE probe is in closer proximity to the heart than with standard transthoracic echo techniques and does not suffer interference from structures such as ribs or lungs being located between the probe and heart. However, there are circumstances in which TEE image quality is suboptimal such as in patients with hiatal hernias wherein the TEE probe cannot adequately contact the esophagus, and prolonged procedures in which the ultrasound gel dissipates and adequate contact with the esophageal wall is lost and image quality is reduced. Thus, there are a variety of procedures that would benefit from improved contact between the TEE probe and esophageal wall. Accordingly, there is a need for a product that enhances and sustains a fluid interface between a TEE probe tip and the esophageal wall for maximizing the quality of diagnostic TEE imaging.

### SUMMARY OF THE INVENTION

The following provides a summary of certain exemplary embodiments of the present invention. This summary is not an extensive overview and is not intended to identify key or critical aspects or elements of the present invention or to delineate its scope.

In accordance with one aspect of the present invention, a first cover for a probe having an ultrasound emitting tip is provided. This probe cover includes an inflatable portion, wherein the inflatable portion further includes a first reservoir, wherein the first reservoir is adapted to fit over the ultrasound emitting tip of the probe, and wherein the first reservoir is adapted to receive a first predetermined volume of fluid; a second reservoir adjacent to the first reservoir, wherein the second reservoir is adapted to receive a second predetermined volume of fluid; and a divider for completely separating the first reservoir from the second reservoir; and a collar connected to or formed integrally with the inflatable portion, wherein the collar is operative to seal the inflatable portion against the probe.

In accordance with another aspect of the present invention, a second cover for a probe having an ultrasound emitting tip is provided. This probe cover includes an inflatable portion, wherein the inflatable portion further includes a first reservoir, wherein the first reservoir is adapted to fit over the ultrasound emitting tip of the probe, and wherein the first reservoir is adapted to receive a first predetermined volume of fluid; a second reservoir in proximity to the first reservoir, wherein the second reservoir is adapted to receive a second predetermined volume of fluid; a divider for completely separating the first reservoir from the second reservoir; a first fluid conduit connected to the first reservoir; and a second fluid conduit connected to the second reservoir; and a non-inflatable portion, wherein the first and second fluid conduits pass through the non-inflatable portion.

In yet another aspect of this invention, a flexible cover for a transesophageal echocardiography transducer probe having an ultrasound emitting tip is provided. This probe cover includes an inflatable portion, wherein the inflatable portion further includes a first reservoir, wherein the first reservoir is adapted to fit over the ultrasound emitting tip of the probe, and wherein the first reservoir is adapted to receive a first predetermined volume of fluid; a second reservoir in proximity to the first reservoir, wherein the second reservoir is adapted to receive a second predetermined volume of fluid; wherein the first predetermined volume of fluid is less than the second predetermined volume of fluid, and wherein the first and second predetermined volumes of fluid are adjustable independent of one another; a divider for completely separating the first reservoir from the second reservoir; a first fluid conduit connected to the first reservoir; and a second fluid conduit connected to the second reservoir; and a non-inflatable portion, wherein the first and second fluid conduits pass through the non-inflatable portion.

Additional features and aspects of the present invention will become apparent to those of ordinary skill in the art upon reading and understanding the following detailed description of the exemplary embodiments. As will be appreciated by the skilled artisan, further embodiments of the invention are possible without departing from the scope and spirit of the invention. Accordingly, the drawings and associated descriptions are to be regarded as illustrative and not restrictive in nature.

### BRIEF DESCRIPTION OF THE DRAWINGS

The accompanying drawings, which are incorporated into and form a part of the specification, schematically illustrate one or more exemplary embodiments of the invention and, together with the general description given above and detailed description given below, serve to explain the principles of the invention, and wherein:

FIG. 1 is a side view of an exemplary transesophageal echocardiography transducer probe cover in accordance with a first embodiment of the present invention;

FIG. 2 is a side view of the transesophageal echocardiography transducer probe cover of FIG. 1, wherein a transesophageal echocardiography transducer probe has been properly inserted therein; and

FIG. 3 is a side view of an exemplary transesophageal echocardiography transducer probe cover in accordance with a second embodiment of the present invention, wherein a transesophageal echocardiography transducer probe has been properly inserted therein.

### DETAILED DESCRIPTION OF THE INVENTION

Exemplary embodiments of the present invention are now described with reference to the Figures. Reference numerals are used throughout the detailed description to refer to the various elements and structures. Although the following detailed description contains many specifics for the purposes of illustration, a person of ordinary skill in the art will appreciate that many variations and alterations to the following details are within the scope of the invention. Accordingly, the following embodiments of the invention are set forth without any loss of generality to, and without imposing limitations upon, the claimed invention.

The present invention provides two different embodiments of a contact-enhancing condom or cover that fits snugly over one end of a transesophageal echocardiography (TEE) transducer probe. This cover is designed to be filled and inflated with water or saline after the probe has been advanced into the esophagus. The water or saline acts as a coupling agent and provides excellent contact with the esophageal wall while eliminating air, which ultrasonic energy does not effectively penetrate. This “bag of fluid” also expands the viewing window of the TEE transducer probe. At least one port or tube is built into the condom so that fluid may be added or removed, as desired. The fluid interface of this invention provides uninterrupted contact between the TEE transducer probe and the esophageal wall, allowing for maximal diagnostic TEE image quality. This invention is particularly useful in difficult procedures, such as: (i) those used with patients having hiatal hernias; (ii) transcatheter aortic valve replacement (TAVR); (iii) open surgical valve repair, and (vi) percutaneous mitral and tricuspid valve repair or replacement. As a product, this invention would be used in the cardiac operating room; cardiac interventional labs where structural heart cases are performed; and TEE labs when an imager encounters a case wherein a hiatal hernia is suspected as the reason for poor image quality. Advantages of the invention include improving diagnostic quality and expediting imaging acquisition by eliminating difficulty associated with obtaining suitable diagnostic images. In addition, the device can cool an overheating probe. Products based on this invention would be purchased by operating rooms, catheterization laboratories, and echocardiography labs based on the decisions of cardiovascular surgeons, interventional cardiologists, noninvasive cardiologists, or others.

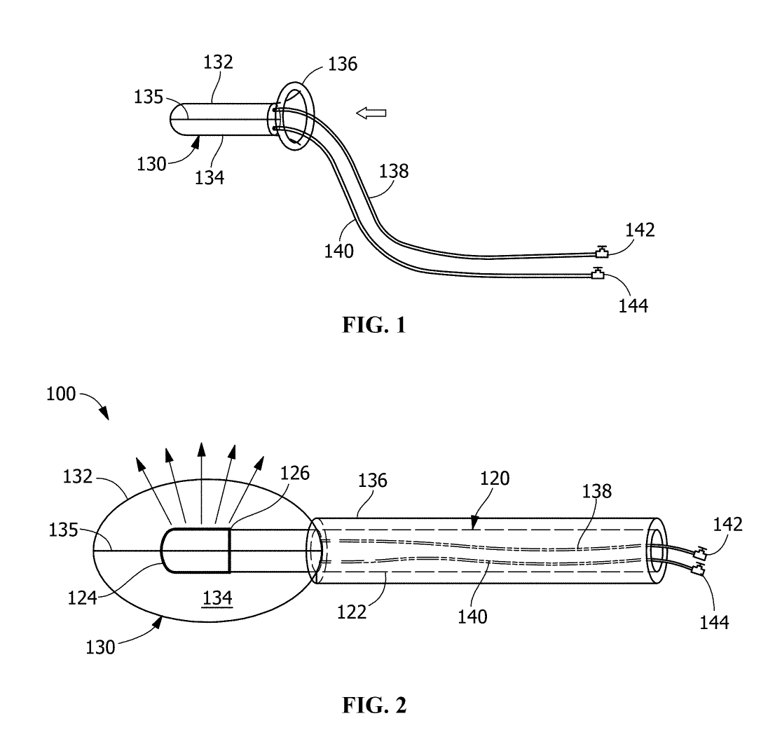

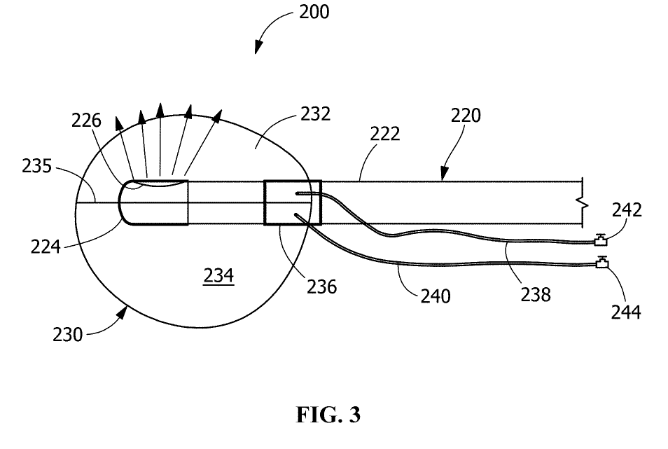

FIGS. 1-2 provide drawings of a first embodiment of this invention wherein the inventive cover extends along the length of the TEE transducer probe, and FIG. 2 provides a drawing of a second embodiment of this invention wherein the inventive cover does not extend along the length of the TEE transducer probe. In the embodiment of FIG. 1, the fluid tubes are mostly internal to the cover and each tube attaches to a three-way stopcock. In the embodiment of FIG. 2, the fluid tubes are mostly external to the cover and each tube also attaches to a three-way stopcock. The inflatable balloon portion of both embodiments is divided, with one side of the balloon holding a smaller volume of fluid. The side of the balloon that contains the smaller volume corresponds to the side of the TEE transducer probe that generates ultrasonic energy.

Again with reference to FIGS. 1-2, a first exemplary TEE transducer and probe cover assembly 100 includes TEE transducer probe 120, which further includes TEE transducer probe handle (body) 122, TEE transducer probe tip 124, and ultrasound emitting portion 126 of TEE transducer probe tip 124. FIG. 1 illustrates the appearance of exemplary TEE transducer probe cover 130 prior to being placed over TEE transducer probe 120 and inflated, and FIG. 2 illustrates the appearance of TEE transducer probe cover 130 after TEE transducer probe 120 has been inserted therein and TEE transducer probe cover 130 has been properly inflated. As best shown in FIG. 2, TEE transducer probe cover includes first (anterior) reservoir portion or balloon 132 and second (posterior) reservoir portion or balloon 134, which accommodates a larger volume of fluid than first reservoir portion 132. First and second reservoir portions 132 and 134, respectively, are separate and distinct from one another and are separated from each other by divider 135. As shown in FIG. 2, sleeve portion 136 extends along the length of TEE transducer probe handle (body) 122. First fluid conduit 138 passes through sleeve portion 136 and is connected to first reservoir portion 132. First fluid conduit 138 is used to introduce fluid into or remove fluid from first reservoir portion 132 and first three-way stopcock 142 is mounted on first fluid conduit 138 for opening and closing first fluid conduit 138. Second fluid conduit 140 passes through sleeve portion 136 and is connected to second reservoir portion 134. Second fluid conduit 140 is used to introduce fluid into or remove fluid from second reservoir portion 134 and second three-way stopcock 144 is mounted on second fluid conduit 140 for opening and closing second fluid conduit 140.

Again with reference to FIG. 2, a second exemplary TEE transducer and probe cover assembly 200 includes TEE transducer probe 220, which further includes TEE transducer probe handle (body) 222, TEE transducer probe tip 224, and ultrasound emitting portion 226 of TEE transducer probe tip 224. FIG. 2 illustrates the appearance of exemplary TEE transducer probe cover 230 after TEE transducer probe 220 has been inserted therein and TEE transducer probe cover 230 has been properly inflated. TEE transducer probe cover includes first (anterior) reservoir portion or balloon 232 and second (posterior) reservoir portion of balloon 234, which accommodates a larger volume of fluid than first reservoir portion 232. First and second reservoir portions 232 and 234, respectively, are separate and distinct from one another and are separated from each other by divider 235. Collar 236 seals TEE transducer probe cover 230 against TEE transducer probe handle (body) 222. First fluid conduit 238 passes through collar 236 and is connected to first reservoir portion 232. First fluid conduit 238 is used to introduce fluid into or remove fluid from first reservoir portion 232 and first three-way stopcock 242 is mounted on first fluid conduit 238 for opening and closing first fluid conduit 238. Second fluid conduit 240 passes through collar 236 and is connected to second reservoir portion 234. Second fluid conduit 240 is used to introduce fluid into or remove fluid from second reservoir portion 234 and second three-way stopcock 244 is mounted on second fluid conduit 240 for opening and closing second fluid conduit 240.

The probe covers of the present invention are used according to the following general method. After removal from the protective package, a small amount of sterile ultrasound gel is placed inside the probe cover at its end. The probe cover is then placed over the tip of the TEE probe orienting the smaller balloon anteriorly and the larger balloon posteriorly, and the cover is then be advanced proximally. Once fully extended, a plastic clip secures the probe cover and tubes (conduits) in place. A three-way stopcock is attached to a connector on each tube. 10 cc syringes filled with water are then attached to each stopcock. The balloons are inflated and then deflated to check the integrity of the system. The exterior of the probe cover should be lubricated with sterile gel. The TEE probe is now ready for insertion. Once situated in the mid esophagus where imaging is desired, the anterior balloon is gently inflated with 5 cc of fluid (or other appropriate volume). The posterior balloon is then inflated gently with up to 10 cc of fluid (or other appropriate volume) until adequate esophageal wall contact is established. Once the desired amount of fluid has been injected, the stopcocks are rotated to prevent backflow into the syringes. The probe can be rotated within the probe cover, as needed; however significant movement cephalad will potentially move the probe tip outside of the balloons' field. Prior to repositioning of the probe distally or cephalad, the balloons should be deflated and the probe and probe cover moved in unison and then re-inflated when the probe has reached the new location. Prior to removal of the TEE probe and probe cover, the balloons should be maximally deflated by withdrawal of fluid by negative suction from the syringes and the stopcocks then rotated to avoid any fluid getting back into the balloons. The probe cover should then be appropriately discarded.

While the present invention has been illustrated by the description of exemplary embodiments thereof, and while the embodiments have been described in certain detail, it is not the intention to restrict or in any way limit the scope of the appended claims to such detail. Additional advantages and modifications will readily appear to those skilled in the art. Therefore, the invention in its broader aspects is not limited to any of the specific details, representative devices and methods, and/or illustrative examples shown and described. Accordingly, departures may be made from such details without departing from the spirit or scope of the general inventive concept.

Additionally, the section headings herein are provided for consistency with the suggestions under 37 C.F.R. 1.77 or otherwise to provide organizational cues. These headings shall not limit or characterize the invention(s) set out in any claims that may issue from this disclosure. A description of a technology in the “Background” is not to be construed as an admission that certain technology is prior art to any embodiment(s) in this disclosure. Neither is the “Summary” to be considered as a characterization of the embodiment(s) set forth in issued claims. Furthermore, any reference in this disclosure to “invention” in the singular should not be used to argue that there is only a single point of novelty in this disclosure. Multiple embodiments may be set forth according to the limitations of the multiple claims issuing from this disclosure, and such claims accordingly define the embodiment(s), and their equivalents, that are protected thereby. In all instances, the scope of such claims shall be considered on their own merits in light of this disclosure, but should not be constrained by the headings set forth herein.

What is claimed:

1. A cover for a probe having an ultrasound emitting tip, comprising:
(a) an inflatable portion, wherein the inflatable portion further includes:
(i) a first reservoir, wherein the first reservoir is adapted to fit over the ultrasound emitting tip of the probe, and wherein the first reservoir is adapted to receive a first predetermined volume of fluid;
(ii) a second reservoir adjacent to the first reservoir, wherein the second reservoir is adapted to receive a second predetermined volume of fluid; and
(iii) a divider for completely separating the first reservoir from the second reservoir; and
(b) a collar connected to or formed integrally with the inflatable portion, wherein the collar is operative to seal the inflatable portion against the probe.

2. The probe cover of claim 1, further comprising a first fluid conduit connected to the first reservoir.

3. The probe cover of claim 2, further comprising a first stopcock connected to the first fluid conduit.

4. The probe cover of claim 1, further comprising a second fluid conduit connected to the second reservoir.

5. The probe cover of claim 4, further comprising a second stopcock connected to the second fluid conduit.

6. The probe cover of claim 1, wherein the first predetermined volume of fluid is less than the second predetermined volume of fluid.

7. The probe cover of claim 1, wherein the first and second predetermined volumes of fluid are adjustable independent of one another.

8. A cover for a probe having an ultrasound emitting tip, comprising:
(a) an inflatable portion, wherein the inflatable portion further includes:
(i) a first reservoir, wherein the first reservoir is adapted to fit over the ultrasound emitting tip of the probe, and wherein the first reservoir is adapted to receive a first predetermined volume of fluid;
(ii) a second reservoir in proximity to the first reservoir, wherein the second reservoir is adapted to receive a second predetermined volume of fluid;
(iii) a divider for completely separating the first reservoir from the second reservoir;
(iv) a first fluid conduit connected to the first reservoir; and
(v) a second fluid conduit connected to the second reservoir; and
(b) a non-inflatable portion, wherein the first and second fluid conduits pass through the non-inflatable portion.

9. The probe cover of claim 8, further comprising a first stopcock connected to the first fluid conduit and a second stopcock connected to the second fluid conduit.

10. The probe cover of claim 9, wherein the first and second stopcocks are three-way stopcocks.

11. The probe cover of claim 8, wherein the first predetermined volume of fluid is less than the second predetermined volume of fluid.

12. The probe cover of claim 8, wherein the first and second predetermined volumes of fluid are adjustable independent of one another.

13. The probe cover of claim 8, wherein the non-inflatable portion includes a sleeve that extends along the length of the probe.

14. The probe cover of claim 8, wherein the non-inflatable portion includes a collar that seals the inflatable portion against the probe.

15. The probe cover of claim 8, wherein the fluid that the reservoirs are adapted to accept is saline or water.

16. The probe of cover of claim 8, wherein the probe cover is adapted for use with a transesophageal echocardiography transducer probe.

17. A flexible cover for a transesophageal echocardiography transducer probe having an ultrasound emitting tip, comprising:
(a) an inflatable portion, wherein the inflatable portion further includes:
(i) a first reservoir, wherein the first reservoir is adapted to fit over the ultrasound emitting tip of the probe, and wherein the first reservoir is adapted to receive a first predetermined volume of fluid;
(ii) a second reservoir in proximity to the first reservoir, wherein the second reservoir is adapted to receive a second predetermined volume of fluid;
a) wherein the first predetermined volume of fluid is less than the second predetermined volume of fluid, and
b) wherein the first and second predetermined volumes of fluid are adjustable independent of one another;
(iii) a divider for completely separating the first reservoir from the second reservoir;
(iv) a first fluid conduit connected to the first reservoir; and
(v) a second fluid conduit connected to the second reservoir; and
(b) a non-inflatable portion, wherein the first and second fluid conduits pass through the non-inflatable portion.

18. The probe cover of claim 17, further comprising a first stopcock connected to the first fluid conduit and a second stopcock connected to the second fluid conduit, wherein the first and second stopcocks are three-way stopcocks.

19. The probe cover of claim 17 wherein the non-inflatable portion includes a sleeve that extends along the length of the probe.

20. The probe cover of claim 17, wherein the non-inflatable portion includes a collar that seals the inflatable portion against the probe.
